(19) United States
(12) Patent Application Publication
(10) Pub. No.: US 2008/0037703 A1
(43) Pub. Date: Feb. 14, 2008

(54) Three dimensional breast imaging

(75) Inventor: Rulei Ting, Holmdel, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: DigiMD Corporation, Middletown, NJ (US)

(21) Appl. No.: 11/502,226

(22) Filed: Aug. 9, 2006

(51) Int. Cl. A61B 6/04

(52) U.S. Cl. 378/37

(57) ABSTRACT

Apparatuses, methods and systems for performing X-ray CT and/or tomosynthesis imaging in connection with mammography. In one such apparatus, a radiation source includes a distal end and a proximal end, as well as an anode operatively connected to a proximally extending shaft that does not substantially extend distally from the anode, and a cathode. The apparatus also includes a radiation receiver located opposite the radiation source such that at least a portion of radiation from the source passes through a patient's breast that is located between the source and receiver. The radiation source and radiation receiver are mutually fixed in relative position as a gantry unit that is capable of rotation about the patient's breast and around an axis of rotation.

### FIELD OF THE INVENTION

The present invention relates to X-ray imaging. More particularly, the present invention relates to Computed Tomography (CT) or tomosynthesis-implemented mammography.

### BACKGROUND

Mammography can be an invaluable tool for the early detection and diagnosis of breast tumors and the like. Unfortunately, conventional mammography has shortcomings that often limit its usefulness. For example, in some circumstances there are inherent image quality issues that arise when compressing the three-dimensional features of the human breast into a conventional two-dimensional mammogram image. Features that are displaced at varying depths within the breast tissue may appear as overlapped in the two-dimensional image, thereby rendering each individual feature difficult to detect or analyze. Conventional mammography typically also has difficulty adequately creating an image of features near a patient's chest wall without disadvantageous X-ray exposure to the patient's chest cavity. In addition, many patients find conventional mammograms to be very uncomfortable, and as a result some patients may not receive mammogram screenings as often as recommended because the patients do not want to experience the discomfort associated with the procedure.

Typical solutions to these problems may involve the use of digital X-ray imaging techniques, such as digital full field flat panel detector mammography and tomosynthesis, but these solutions also have numerous shortcomings. For example, often these solutions still suffer from image compression problems, which adversely affect image quality and the image's diagnostic value. This is even generally true for tomosynthesis-based mammograms, despite the imaging improvement provided by tomosynthesis' ability to enhance features at a given depth within a breast by processing digital images obtained at several projection angles.

X-ray Computed Tomography (CT) systems have been proven to be invaluable in medical imaging since their invention in the 1970s. With such a system, clean cross-sectional images of human anatomy can be generated, each at a predetermined or desired depth within a patient. Modern X-ray CT systems are capable of producing many cross-sectional images at the same time. These images can be processed and enhanced to reconstruct three-dimensional structures of a human body. X-ray CT has found many applications in medical imaging, except for general, wide-spread breast imaging, which has proven to be a difficult application in which to apply conventional X-ray CT.

For example, conventional commercial X-ray CT typically does not provide the necessary spatial resolution for breast imaging, and still exposes additional regions in a patient to high levels of X-ray radiation if a complete image of the breast is to be acquired. In addition, the act of acquiring a prior art X-ray CT image is cumbersome. For example, imaging typically occurs while patients are lying face down in a horizontal position with a breast placed in a cavity that is in the active X-ray imaging area. Such a system is bulky, difficult to operate and is prone to patient motion artifacts. Further, such a system is typically very large, which poses infrastructure problems for the medical offices in which the conventional X-ray CT system is to be installed.

### SUMMARY

Apparatuses, methods and systems for performing X-ray CT and/or tomosynthesis imaging in connection with mammography are provided. Various embodiments of an imaging apparatus may include, for example, an X-ray gantry having a small bore opening for receiving a human breast while a patient is in a standing position. Embodiments of the apparatus may also include an anti scatter grid for use in connection with mammography-related tomosynthesis. Further embodiments may include breast holders for immobilizing a breast to be imaged.

Relate methods may involve placing a patient's breast in a bore opening of a vertically-oriented gantry that includes an X-ray radiation source and an X-ray radiation detector that are located at opposing positions of the gantry. The method may also involve immobilizing the patient's breast within the bore opening while the patient is in a seated or standing position, capturing an image of the patient's breast and releasing the patient's breast from the bore opening.

### BRIEF DESCRIPTION OF THE DRAWINGS

The foregoing Summary, as well as the following detailed description of the various embodiments, is better understood when read in conjunction with the appended drawings. For the purpose of illustrating the slot embodiments, there is shown in the drawings example constructions of various embodiments; however, the present invention is not limited to the specific methods and instrumentalities disclosed. In the drawings:

FIG. 1A illustrates a prior art X-ray source and detector;

FIG. 1B is a schematic of an example X-ray source and detector configuration;

FIGS. 2A-B are schematics of another example X-ray source and detector configuration;

FIG. 3 illustrates an example X-ray gantry;

FIGS. 4A-B illustrate example breast holders; and

FIG. 5 is a flowchart illustrating an example method of X-ray imaging.

### DETAILED DESCRIPTION

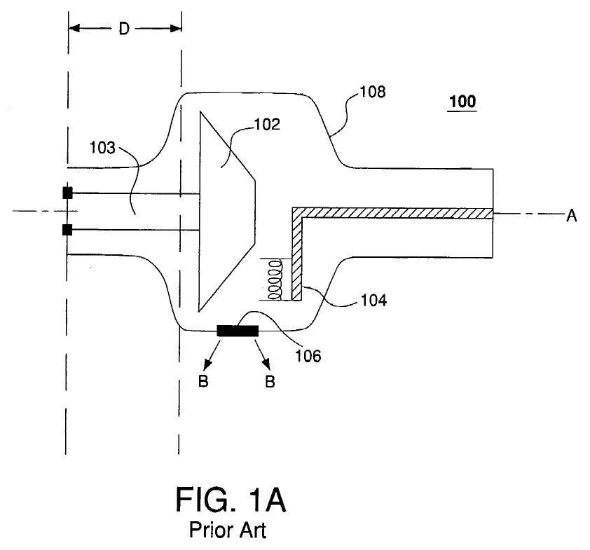

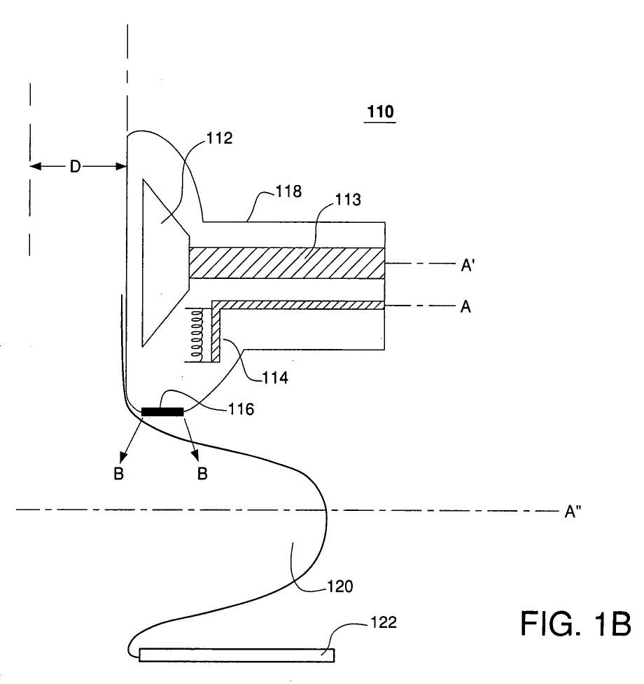

Various apparatuses, methods and systems for performing X-ray CT or tomosynthesis imaging in connection with mammography are provided. For purposes of explanation and comparison, a conventional X-ray source is first described herein. Referring now to FIG. 1A, conventional X-ray source 100 includes anode 102, which is coupled to shaft 103, and cathode 104. Anode 102, shaft 103 and cathode 104 are arranged along axis A. Electrons from cathode 104 are accelerated by an electric field induced by an applied voltage and bombard the surface of anode 102. X-ray radiation is then generated, and the radiation escapes conventional X-ray source 100 by way of X-ray output window 106 in housing 108, as indicated by arrows B, and then passes through an object to be analyzed. Anode 102 is typically fabricated from materials with a high atomic number, such as Molybdenum or Tungsten. To prevent excess heat generated at the same spot in the anode 102, anode 102 is rotated around axis A using shaft 103. As can be seen in FIG. 1A, a conventional design for X-ray source 100 provides for shaft 103 to extend a distance D beyond the side of anode 102 facing in a direction away from cathode 104. As a result, a patient who is standing to the left of X-ray source such that the patient's breast is placed under X-ray output window 106 cannot have her chest wall and base of her breast (not shown in FIG. 1A for clarity) any closer to X-ray source 100 than distance D, thus rendering a conventional X-ray source often unstable or difficult for mammograms of the entire breast.

Referring now to FIG. 1B, a cross-sectional view of example X-ray source 110 illustrating one embodiment of the present invention includes anode 112 and its associated shaft 113, to which anode 112 is operatively coupled, which together rotate about axis A′. In addition, cathode 114 is aligned with axis A. Axes A′ and A may be offset, which may be compared to FIG. 1A, where anode 102, shaft 103 and cathode 104 may be all aligned with axis A. Electrons from cathode 114 are accelerated by an electric field induced by an applied voltage and bombard the surface of anode 112. X-ray radiation is then generated, and the radiation escapes X-ray source 110 by way of X-ray output window 116 in housing 118, as indicated by arrows B, and then passes through an object to be analyzed. The X-ray radiation is then detected by detector 122, which may convert the detected X-ray radiation into a signal that may be converted by imaging hardware and/or software (not shown in FIG. 1B) into an image.

It can be seen that shaft 113 now extends away from the patient's chest wall and in the same direction cathode 114 extends to allow anode 112 to come closer to the human chest wall (represented in FIG. 1B as part of target breast 120). As a result, anode 112 and output window 116 may be located close to a patient's chest wall and closer than conventional X-ray source 100 of FIG. 1A by about distance D. In many conventional CT systems, the relationship among shaft 113, cathode 114, and anode 113 as shown in FIG. 1B is not necessary because the entire object being imaged can be placed inside the opening bore of the CT device (i.e., in an area below the X-ray output window). In CT mammography, however, placing the entire patient in the bore opening causes potentially harmful X-ray exposure to the patient's chest cavity. Thus, the configuration of FIG. 1B allows the bore opening of a CT device to be small enough to receive a patient's breast rather than the entire patient, while still permitting imaging of regions in the vicinity of the patient's chest wall. To obtain high spatial resolution, monolithic detectors may be employed as detector 122 in an embodiment.

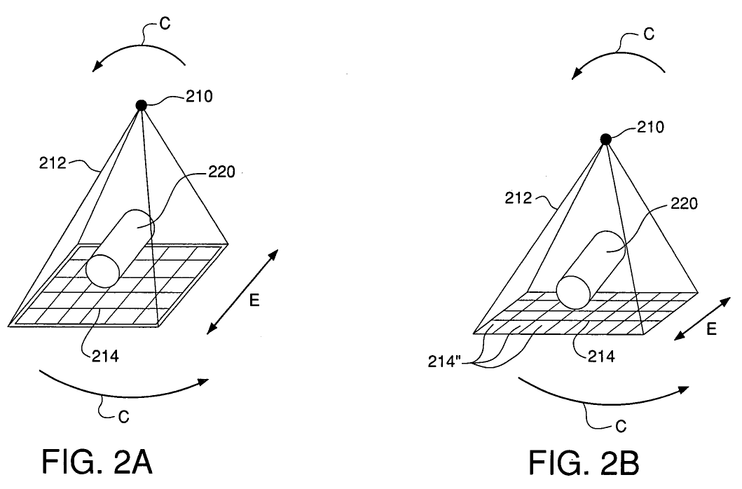

FIGS. 2A-B illustrate example configurations of X-ray source 210 and detector 214. A housing, which may be disposed around X-ray source 210 and detector 214 (such as housing 118 of FIG. 1B) is omitted for clarity. FIG. 2A illustrates an example X-ray source 210 and detector 214 configuration in accordance with one embodiment. X-ray source 210 (which may be, for example, X-ray source 110 as was discussed above in connection with FIG. 1B) emits X-rays (the effective outer boundaries of which are represented by lines 212) that pass through object 220 and are received by detector 214. Detector 214 may be a single monolithic flat panel detector such as a-Si couple with scintillation materials, or the direct conversion directors, such as, for example, Se flat panels.

Detector 214 generates a signal that can be used by imaging software and/or hardware to generate an image (e.g., CT or tomosynthesis) of object 220, or a region of interest anywhere in object 220. Such hardware or software may be of any type, such as conventional systems. To generate CT images of object 220, X-ray source 210 and detector 214 may rotate around object 220 as indicated by arrows C. Rotation may be clockwise or counter-clockwise.

While an embodiment may be employed in connection with CT imaging, embodiments may also be employed in connection with the acquisition of other types of X-ray images. For example, the configuration illustrated in FIG. 2A may be used in connection with tomosynthesis, where X-ray source 210 and detector 214 rotate around object 220, as indicated by arrows C, with limited angular coverage. Such limited angular coverage may be, for example, ±30 degrees.

The configuration depicted in FIG. 2A may perform both CT imaging and tomosynthesis as different scanning modes. For example, when performing CT imaging, the configuration illustrated in FIG. 2A may rotate around object 220 using approximately 180 degrees or more of rotation. When performing tomosynthesis, the configuration depicted in FIG. 2A may rotate around object 220 using the aforementioned ±30 degrees or the like. Alternatively, the configuration depicted in FIG. 2A may be constructed such that only CT imaging or tomosynthesis may be performed. In such an embodiment, a cost savings may be realized because the resulting device need not be capable of performing both imaging methods, which may reduce the number or complexity of parts needed for such a device, for example. When the configuration depicted in FIG. 2A is used in connection with tomosynthesis, the fixed relative position between X-ray source 210 and detector 214 during the scanning enables the introduction of an anti-scatter grid proximate detector 214 (anti-scatter grid not shown in FIG. 2A for clarity). The anti-scatter grid may be of any type.

The configuration depicted in FIG. 2A may be adapted to capture images of object 220 in any number of ways. For example, the X-ray active region (i.e., the region that is irradiated by X-ray source 210 and may be detected by detector 214) may be equal to or longer than the breast length. In such a situation, X-ray source 210 and detector 214 may only need to rotate, as indicated by arrow C, to capture an image because detector 214 has a length along direction E that is longer than the target breast. Alternately, the X-ray active region may be shorter than the breast length (which is typically referred to in conventional applications as a “small cone-angle CT system”). To perform mammography using such a small cone angle system, X-ray source 210 and detector 214 may be moved as close as possible to, for example, the human chest wall and then may be rotated as indicated by arrows C in FIG. 2A, and also translated from the chest wall as indicated by arrow E while the breast is fixed in place. Alternatively, X-ray source 210 and detector 214 may be translated towards the chest wall during rotation. The rotation and translation may occur simultaneously, thereby forming a helical pattern of movement on the part of X-ray source 210 and detector 214. Alternatively, rotation may occur at a fixed distance from a patient's chest wall, and translation may occur once an image has been captured at the fixed distance, thereby forming a pattern of movement of X-ray source 210 and detector 214 that resembles a series of rings that progressively move away from (or toward) a patient's chest wall.

FIG. 2B illustrates another example configuration of X-ray source 210 and detector 214 in accordance with an embodiment. In the configuration depicted in FIG. 2B, detector 214 is formed by a plurality of modular detectors 214′. Each discrete modular detector 214′ may comprise, for example, a two-dimensional array of detector pixels. The detector pixel size may be, for example, 50-100 μm². Modular detectors 214′ may be, for example, a direct conversion type of detector such as CZT (CdZnTe), smaller area Se scintillator coupled to small area a-Si, CMOS diode, or other.

Modular detectors 214′ may be arranged such that detector 214 can cover enough area for breast CT imaging for the particular mode of collecting an image information. Again, such rotation may be clockwise or counter-clockwise. Thus, when X-ray source 210 and detector 214 rotate around object 220 in direction C, the modular detectors 214′ of detector 214 are able to gather X-ray radiation that has passed through object 220 to generate an image. The X-ray active region of the configuration of FIG. 2B may be smaller than the length of a breast to be analyzed, in which case an image may be acquired using translation in a direction E as was discussed above in connection with FIG. 2A.

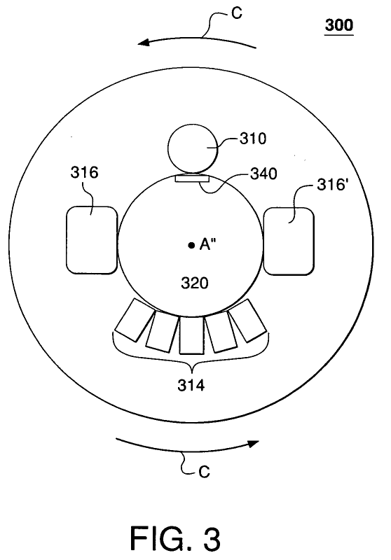

FIG. 3 illustrates a cross-sectional view of a structure that may be used according to the configuration illustrated in FIG. 2A-B, for example. Gantry 300 may include X-ray source 310, such as X-ray source 110 as was discussed above in connection with FIG. 1B. In addition, gantry 300 may include high voltage power 316 and 316′, detector 314 and bore opening 320. Detector 314 may be a single large-field detector, or may be formed from a plurality of modular detectors, as was discussed above in connection with FIGS. 2A-B. FIG. 3 illustrates detector 314 as formed from a plurality of modular detectors, but such illustration is merely for purposes of explanation. Gantry 300 may be adapted to rotate, as indicated by arrows C, around axis A″. Gantry 300 may also be adapted to translate laterally along axis A, which is perpendicular to the plane shown in FIG. 3. The exact number and placement of components 310, 314, 316 and 316′ may be determined by operational requirements, and therefore the present invention is not limited to the configuration depicted in FIG. 3.

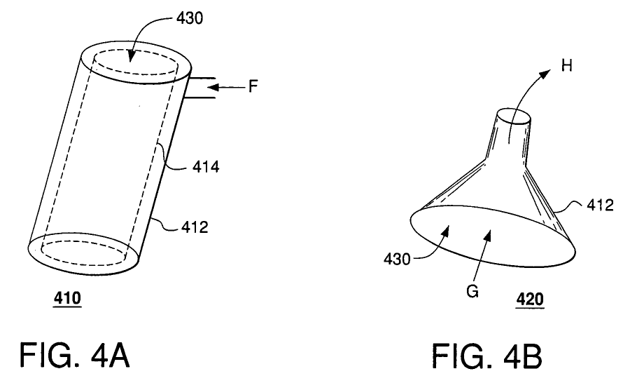

Gantry 300 may be vertically oriented (i.e., the gantry is positioned substantially vertically such that axis A″, about which gantry 300 rotates, is oriented substantially horizontally). Thus, a patient may remain in a standing position and be positioned proximate gantry 300 such that a breast may be placed within bore opening 320. A housing around gantry 300 may be stationary or a component (not shown in FIG. 3) may be coupled to gantry 300- or to a CT/tomosynthesis system of which gantry 300 is a part—to remain substantially stationary and in contact with a patient's breast while gantry 300 rotates. Thus, chafing and other forms of patient discomfort may be avoided. For example, a breast holder may be employed to hold the breast to be examined within bore opening 320. Also not shown in FIG. 3 are any control and image processing units that may be associated and/or operatively coupled to gantry 300 as part of a CT or tomosynthesis system of which gantry 300 may be a part. Example breast holders will be discussed below in connection with FIGS. 4A-B.

As noted above, gantry 300 may rotate around axis A″. The angle of rotation that gantry 300 may undergo may be determined by the type of imaging to be acquired. For example, if CT images are to be acquired, an angle of rotation of approximately 180 degrees or more may be used. If tomosynthesis images are to be acquired, the angle of rotation may be less than 180 degrees. The present invention is not limited to any particular extent of rotation.

Preferably, bore opening 320 may be approximately 30 cm in diameter, or otherwise sized to adequately receive a human breast and any associated equipment, such as a breast holder, rather than sized to receive an entire human body. Gantry 300 may be oriented vertically, such that bore opening 320 is adapted to receive a breast of a patient who is in a standing or seated position. Power and data traffic to electronic components within gantry 300 may be fed through, for example, slip rings, which may be conventional.

In one embodiment, gantry 300 may be attached to a system fixture, such as a housing for an X-ray imaging device of which gantry 300 is a part (not shown in FIG. 3 for clarity), such that there is substantially no relative motion between gantry 300 and the system fixture. In such an embodiment, the active X-ray region in a direction perpendicular to axis A″ (e.g., a straight-line path between X-ray source 310 and detector 314) and detector 314 may be large enough to cover the object to be analyzed (e.g., a human breast).

It will be appreciated that while such an embodiment may result in a simpler—and likely less expensive—gantry 300 and associated X-ray imaging device, the large cone-angle of the X-ray beam may adversely affect the accuracy of image reconstruction. The present invention encompasses employing advanced three-dimensional reconstruction algorithms, which may be conventional, that may alleviate such effects.

An X-ray slit 340 may be placed near the X-ray tube. Slit 340 may limit the active X-ray region to a smaller size. As a result, only a section of the object to be analyzed is imaged by a small portion of the detector 314 area. Slit 340 may then be moved, either manually or automatically, to another position for imaging another section of the object. This sequence may be repeated until the entire object—or region of interest in the object—is imaged.

In one embodiment, the X-ray active region may be as long as or longer than the length of the object to be analyzed. In such an embodiment, gantry 300 need only rotate around axis A, as indicated by arrows C, to image the object. In an alternate embodiment, the X-ray active region may be shorter than the length of the object to be analyzed. In such an embodiment, gantry 300 may rotate around axis A as well as translate along axis A as was noted above in the discussion of small cone-angle X-ray CT imaging in FIG. 2A.

In another alternate embodiment involving small cone-angle X-ray CT, the object to be analyzed (or, for example, a patient) may be placed on a moving platform. In such an embodiment gantry 300 need not translate during the data collection. Instead, the platform/patient may move away in unison from gantry 300 along axis A while gantry 300 rotates around axis A to form the same effect as the helical scanning motion discussed above in connection with small cone-angle X-ray CT.

Data collection may be performed in such a way that the relative position between the object to be analyzed and gantry 300 is fixed during data collection. With the completion of data collection at this position, the relative position between the object and gantry 300 may be shifted to cover another part of the object. This sequence may be repeated until the entire object or region of interest within the object is covered.

A breast holder may be incorporated into the design of an X-ray imaging device in which gantry 300 is employed. A breast holder serves to assist in immobilizing a breast to be analyzed, which may be helpful in the production of high quality CT or tomosynthesis images because accurate relative positioning between gantry 300 and the breast can be maintained. Immobilizing the breast may diminish degradation in spatial resolution and image artifacts that tend to degrade the quality of a resulting image. A breast holder preferably remains substantially stationary relative to the patient, even in situations where gantry 300 rotates to acquire an image. Preferably, a breast holder should be coupled to a gantry 300—and/or the CT/tomosynthesis system of which gantry 300 is a part—to enable gantry 300 to rotate while the breast holder remains substantially stationary. In embodiments where gantry 300 translates along axis A, the breast holder may translate with gantry 300, may move independently, or may remain stationary.

FIG. 4A illustrates an example breast holder 410. Breast holder 410 has a generally cylindrical shape that defines an outer casing 412, an inner casing 414 and a cavity 430. Cavity 430 may be large enough to hold an object to be analyzed such as, for example, a human breast. Inner material 414 may be fabricated from a flexible and/or expandable material that can substantially enclose the inner surface of outer casing 412 with a substantially air-tight seal. Alternatively, inner material 414 may be a substantially enclosed sleeve that fits against an inner surface of outer casing 412. Breast holder 410 may include rigid outer casing 412, which may be fabricated from materials that X-rays can penetrate without significant loss of intensity. Such materials typically have low average atomic number, such as plastic, carbon fiber, and the like.

Breast holder 410 may be mounted into a bore opening of a CT system (such as, for example, bore opening 320 of gantry 300 as discussed above in connection with FIG. 3), possibly with a fixture that is connected to a stationary assembly of the CT/tomosynthesis system of which breast holder 410 is a part (fixture not shown in FIG. 4A for clarity). The fixture should be outside the active X-ray region to avoid object-induced image artifacts. Upon the insertion of the object to be analyzed, such as a human breast, into cavity 430, pressurized air or the like may be pumped into inner material 414 (or between inner material 414 and the inside of outer casing 412), as indicated by arrow F, thereby securely enclosing and immobilizing the breast.

The amount of pressure to place on the breast may be predetermined or set manually or automatically. It will be appreciated that such an amount of pressure should be sufficient to uniformly squeeze the breast to the point that the breast is substantially immobilized with respect to the CT device, but not so great that the pressure is uncomfortable for the patient. After the proper pressure is reached, the breast holder may move slightly along an axis (such as, for example, axis A as discussed above in connection with FIG. 3) to pull the breast towards the scanner (i.e., away from the patient). This hold-and-pull method may ensure that the breast does not move during imaging while also enabling the CT system to cover breast tissue that is close to the patient's chest wall. A disposable or reusable liner may be used to prevent the breast from contacting breast holder 410, for sanitation or other purposes. Alternatively, all or part of breast holder 410 may be disposable.

To avoid the loss in dose efficiency that would take place if holder 410 absorbed excessive amounts of X-ray radiation, materials from which holder 410 is fabricated may be selected such that all or part of holder 410 is substantially X-ray transparent. As a result, the dose penalty to the patient may be minimized.

The CT system can be calibrated with breast holder 410 extracted from the bore opening. The calibration process may include, for example, spectral calibration and air calibration, after which holder 410 may be inserted into the bore opening. Thus, holder 410 and the breast together may act as the object to be scanned by the CT system. However, due to the fact that the breast is always inside the holder, a maximum image field-of-view can be set equal to the inner diameter of holder 410, thus excluding the periphery of outer casing 412 of holder 410 from the resulting image.

FIG. 4B illustrates another example breast holder 420. Breast holder 420 may include a rigid outer casing 412 that is formed in the shape of a cone, for example. An object to be analyzed such as, for example, a human breast, may be placed within cavity 430. A breast may be inserted into cavity 430 as indicated by arrow G. Like breast holder 410 discussed above in connection with FIG. 4A, breast holder may have a disposable or reusable liner (not shown in FIG. 4B for clarity).

A partial vacuum may be formed by withdrawing air from cavity 430 after the breast has been placed therein, as indicated by arrow H, to hold the breast in place within cavity 430. In addition, the partial vacuum may serve to slightly pull the breast away from the patient's chest wall to enable imaging of the breast in the vicinity of the chest wall, as well as of the chest wall itself. As was the case with breast holder 410 discussed above in connection with FIG. 4A, breast holder 420 may be fabricated from materials that are substantially X-ray transparent.

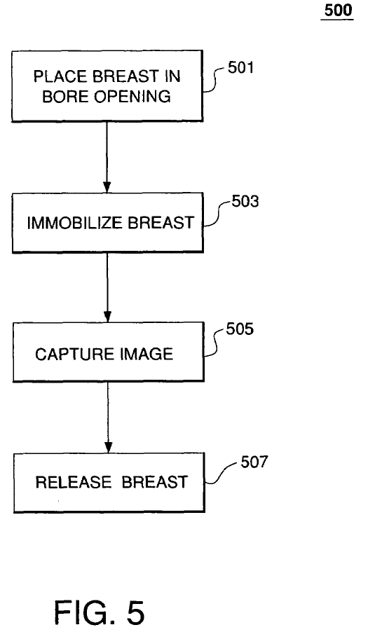

Now that various apparatuses and systems for performing mammography using X-ray CT and/or tomosynthesis imaging have been described, FIG. 5 illustrates an example method 500 of performing such imaging according to one embodiment. At step 501, a patient's breast is placed within a bore opening of, for example, gantry 300 as discussed above in connection with FIG. 3 or other structure discussed above. The patient may be in a standing or seated position because, as noted above, said gantry 300 may be small enough to be oriented vertically.

At step 503, the breast is immobilized using, for example, a breast holder as discussed above in connection with FIGS. 4A-B. During step 503, the breast may also be positioned so as to enable imaging of a region in the vicinity of the patient's chest wall. Such positioning may involve, for example, the pulling away of the breast from the patient's chest wall, as was also discussed above in connection with FIGS. 4A-B. At step 505, an X-ray image of the breast is captured using, for example, any or all of the CT and/or tomosynthesis methods discussed above in connection with FIG. 3. At step 507, at the completion of imaging, the breast may be released from the breast holder/bore opening. It will be appreciated that other steps may be involved with method 500 such as, for example, placing a lining in the breast holder or on the patient's breast, calibration of the X-ray system and the like.

The subject matter of the described embodiments is described with specificity to meet statutory requirements. However, the description itself is not intended to limit the scope of this patent. Rather, the inventor has contemplated that the claimed subject matter might also be embodied in other ways, to include different steps or elements similar to the ones described in this document, in conjunction with other present or future technologies. Therefore, the invention should not be limited to any single embodiment, but rather should be construed in breadth and scope in accordance with the appended claims. Moreover, although the term “step” may be used herein to connote different aspects of methods employed, the term should not be interpreted as implying any particular order among or between various steps herein disclosed unless and except when the order of individual steps is explicitly described.

What is claimed:

1. A mammography apparatus comprising:
a radiation source having a distal end and a proximal end approximately opposite the distal end, the radiation source comprising an anode operatively connected to a proximally extending shaft that does not substantially extend distally from the anode, and a cathode; and
a radiation detector located opposite the radiation source such that at least a portion of radiation from the source passes through a patient's breast that is located between the source and detector, wherein
the radiation source and radiation detector are attached to a gantry unit that is capable of rotation about the patient's breast and around an axis of rotation.

2. The apparatus of claim 1, further comprising an antiscattering grid disposed between the radiation source and the radiation detector.

3. The apparatus of claim 1, wherein the gantry unit is capable of translation in a direction approximately parallel to the axis of rotation, with both the radiation detector and radiation source translating together, or with the radiation detector fixed while the radiation source translates along the axis, or with the radiation source fixed while the radiation detector translates along the axis.

4. The apparatus of claim 3, wherein the rotation of the gantry unit is approximately helical.

5. The apparatus of claim 1, wherein the axis of rotation is such that the patient's breast may be placed between the radiation source and the radiation detector while the patient is in a standing or seated position.

6. The apparatus of claim 1, wherein the radiation detector is comprised of a plurality of detector elements.

7. The apparatus of claim 1, wherein the radiation detector is a large-field X-ray detector.

8. The apparatus of claim 1, wherein the gantry unit is capable of rotating about the patient's breast for more than 180 degrees for complete tomography and less than 180 degrees for tomosynthesis

9. The apparatus of claim 8, wherein the gantry unit is capable of translation in a direction approximately parallel to the axis of rotation of the gantry unit.

10. The apparatus of claim 9, wherein the rotation of the gantry unit is approximately helical.

11. The apparatus of claim 9, further comprising a breast holder that is adapted to translate substantially in unison with the gantry unit but not rotate with the gantry unit.

12. The apparatus of claim 1, further comprising a breast holder that includes an inflatable sleeve, whereby the sleeve is capable of receiving the patient's breast while substantially deflated and encasing the breast upon inflation.

13. The apparatus of claim 12, wherein the breast holder is capable of translation away from the patient's chest to extend the breast.

14. The apparatus of claim 12, wherein the sleeve extends the breast away from the patient's chest upon inflation.

15. The apparatus of claim 1, further comprising a breast holder that includes a rim for contacting the patient and capable of withstanding an internal vacuum that extends the breast away from the patient's chest.

16. The apparatus of claim 1, wherein the radiation detector produces a signal that can be used to generate a Computed Tomography or tomosynthesis image.

17. A method comprising:
placing a patient's breast in a bore opening of a vertically-oriented gantry, wherein the gantry includes an X-ray radiation source and an X-ray radiation detector that are located at substantially opposing positions of the gantry;
immobilizing the patient's breast within the bore opening while the patient is in a seated or standing position;
capturing an image of the patient's breast; and
releasing the patient's breast from the bore opening.

18. The method of claim 17, wherein said placing step further comprises inserting the patient's breast into a breast holder.

19. The method of claim 18, wherein said immobilizing step comprises inflating a liner of the breast holder.

20. The method of claim 18, wherein said immobilizing step comprises pulling the breast by withdrawing air from the breast holder.

21. The method of claim 17, wherein said capturing step generates a tomosynthesis image of at least a portion of the patient's breast.

22. The method of claim 17, wherein said capturing step generates a Computed Tomography image of at least a portion of the patient's breast.

23. A method of performing mammography comprising:
providing an apparatus that comprises a radiation source having a distal end and a proximal end approximately opposite the distal end, wherein the radiation source comprises an anode operatively connected to a proximally extending shaft that does not substantially extend distally from the anode, and a cathode, and wherein the apparatus further comprises a radiation receiver located substantially opposite the radiation source such that at least a portion of radiation from the source passes through a patient's breast that is located between the source and receiver, wherein the radiation source and radiation receiver are attached to the gantry;
positioning the distal end of the radiation source near the patient's chest; and
rotating the gantry around the patient's breast while the source emits radiation that impinges on the receiver.

24. The method of claim 23, wherein the gantry rotates around an axis of rotation, and wherein the method further comprises translating the gantry along the axis of rotation.

25. The method of claim 23, wherein said rotating step further includes rotating the gantry about the patient's breast for more than 180 degrees for complete tomography and less than 180 degrees for tomosynthesis.

26. A breast holder for immobilizing a breast during mammography comprising:
an inflatable sleeve that forms an aperture for receiving a patient's breast during mammography, the sleeve having a first, substantially deflated position during which the patient's breast is insertable and a second, substantially inflated position in which the sleeve is capable of contacting a circumference of the patient's breast, wherein the sleeve is moveable and capable of extending the breast in a direction away from the chest wall of the patient.

27. The breast holder of claim 26, further comprising a substantially rigid outer casing to which the inflatable sleeve is coupled, wherein said outer casing is adapted to be received by a vertically-oriented gantry used in connection with mammography.

28. The breast holder of claim 27, wherein said gantry is adapted to rotate while the breast holder does not rotate.

29. The breast holder of claim 28, wherein said gantry is adapted to rotate around an axis of rotation, and wherein said gantry and breast holder are adapted to translate along the axis of rotation substantially in unison.

30. A breast holder for immobilizing a breast during mammography comprising:
a body that forms an aperture for receiving a patient's breast during mammography, the body having a rim for contacting a patient's trunk and capable of withstanding an internal vacuum for pulling the patient's breast relative to the patient's body, wherein said body is adapted to be received by a vertically-oriented gantry used in connection with mammography.

31. The breast holder of claim 30, wherein said gantry is adapted to rotate while the breast holder does not rotate.

32. The breast holder of claim 31, wherein said gantry is adapted to rotate around an axis of rotation, and wherein said gantry and breast holder are adapted to translate along the axis of rotation substantially in unison.
